(19) United States
(12) Patent Application Publication
(10) Pub. No.: US 2019/0374509 A1
(43) Pub. Date: Dec. 12, 2019

(54) Methods of Using Metamizole for Treatment of Pyrexia in Equines

(71) Applicant: Kindred Biosciences, Inc., Burlingame, CA (US)

(72) Inventors: Emily Sundman, Lipan, TX (US); Melinda Poole O'Banion, Louisburg, KS (US)

(73) Assignee: Kindred Biosciences, Inc., Burlingame, CA (US)

(21) Appl. No.: 16/438,130

(22) Filed: Jun. 11, 2019

Related U.S. Application Data
(60) Provisional application No. 62/683,882, filed on Jun. 12, 2018.
(60) Provisional application No. 62/818,221, filed on Mar. 14, 2019.

(51) Int. Cl. A61K 31/4152; A61K 9/00; A61P 29/00

(52) U.S. Cl. CPC A61K 31/4152; A61P 29/00; A61K 9/0019

(57) ABSTRACT

Methods for treating pyrexia and other diseases in equines involving administering a parenteral formulation containing metamizole are disclosed.

### CROSS-REFERENCE TO RELATED APPLICATIONS

This application claims the benefit of priority of U.S. Provisional Application No. 62/683,882, filed Jun. 12, 2018, and U.S. Provisional Application No. 62/818,221, filed Mar. 14, 2019, each of which is incorporated by reference herein in its entirety for any purpose.

### FIELD

The present disclosure provides new treatment methods for administering metamizole to non-human animals, particularly equines, for therapeutic purposes, including, but not limited to, the treatment of pyrexia.

### BACKGROUND

Pyrexia (fever) is common to all mammals, including equines such as horses. Pyrexia is an elevation in core body temperature above the normal range due to an increase in the temperature regulatory set-point. Pyrexia can be caused by many medical conditions ranging from non-serious to potentially serious and even life threatening. These include viral, bacterial, and parasitic infections, such as equine encephalomyelitis, equine influenza, equine herpes virus, West Nile virus, strangles, and Potomac horse fever. Physical trauma, immune-mediate causes, and stress can also cause a fever in a horse. While a fever can be a useful defense mechanism, since the body's immune response can be strengthened at higher temperatures, very high body temperatures, particularly for prolonged periods of time, can pose significant health risks to the patient. Elevated temperatures may also lead to secondary health issues in the animal; including inappetance, that pose additional health risks. In the setting of animal and human healthcare, it often becomes necessary to reduce a patient's temperature to a safer range (e.g., treat the patient's fever) by medical intervention. Some of the most common causes of febrile illness in horses are communicable diseases (such as respiratory infections), certain forms of colic, post-vaccination reactions, and tick- and mosquito-borne infections.

Dipyrone is a non-opioid, nonsteroidal anti-inflammatory drug (NSAID) that is approved and on the market for use in humans and animals, including horses, outside of the United States. While well-known as dipyrone within the United States, internationally the medication is identified as metamizole or metamizole sodium (international non-proprietary name). Currently, there are no Food and Drug Administration (FDA)-approved dipyrone medications available to veterinarians in the United States.

Methods for using and administering metamizole to equines are provided by the present disclosure.

### SUMMARY

The present disclosure provides methods for the treatment of pyrexia and other conditions in equines by administration of a therapeutically effective amount of metamizole. In some embodiments, the methods include periodically administering a therapeutically effective amount of metamizole by parenteral administration to an equine. The present disclosure further includes, in embodiments, various methods of treatment, including, but not limited to, dosage regimens.

In some embodiments, the present disclosure provides methods for treating an equine with pyrexia. In some embodiments, the methods comprise administering a therapeutically effective dose of metamizole to an equine, wherein the administration is repeated at least once after an interval of about 12 hours.

In some embodiments, the dose is in the range of 20 mg metamizole per kg of equine subject weight (mg/kg) to 50 mg/kg, or in the range of 20 mg metamizole per kg of equine subject weight (mg/kg) to 40 mg/kg, or in the range of 20 mg metamizole per kg of equine subject weight (mg/kg) to 30 mg/kg, or in the range of 25 mg metamizole per kg of equine subject weight (mg/kg) to 35 mg/kg, or in the range of 25 mg metamizole per kg of equine subject weight (mg/kg) to 30 mg/kg, or in the range of 30 mg metamizole per kg of equine subject weight (mg/kg) to 35 mg/kg, or in the range of 27 mg metamizole per kg of equine subject weight (mg/kg) to 33 mg/kg, or in the range of 28 mg metamizole per kg of equine subject weight (mg/kg) to 32 mg/kg. In an embodiment, the dose is 30 mg metamizole per kg of equine subject weight (mg/kg).

In some embodiments, methods are provided wherein the metamizole is administered twice a day for at least 1 day, at least 2 days, at least 3 days, at least 4 days, at least 5 days, at least 6 days, at least 7 days, at least 8 days, or at least 9 days. In some embodiments, the metamizole is administered no more than 5 times in a period of 2 days. In some embodiments, the metamizole is administered no more than 4 times in a period of 2 days. In some embodiments, the metamizole is administered no more than 7 times in a period of 3 days. In some embodiments, the metamizole is administered no more than 6 times in a period of 3 days. In some embodiments, the metamizole is administered to the equine twice a day for 1 to 9 days, for 1 to 8 days, for 1 to 7 days, for 1 to 6 days, for 1 to 5 days, for 1 to 4 days, for 1 to 3 days, or for 1 to 2 days. In some embodiments, the metamizole is administered no more than 18 times in a period of 9 days. In some embodiments, the metamizole is not administered any more than two times per 24-hour period (i.e. no more than twice daily).

In some embodiments, methods are provided wherein the metamizole is administered via intramuscular, intravenous (IV), or subcutaneous administration. In some embodiments, the metamizole is administered via intravenous administration. In some embodiments, the metamizole is administered via slow intravenous administration.

In some embodiments, the dose of metamizole is administered as a pharmaceutical formulation, i.e. the metamizole is comprised within a pharmaceutical formulation that may contain other ingredients. In some embodiments, the concentration of metamizole in the pharmaceutical formulation is from 200 mg/ml to 750 mg/ml or from 400 mg/ml to 600 mg/ml. In embodiments, the concentration of metamizole is 500 mg/ml.

In some embodiments, the metamizole is metamizole sodium monohydrate.

In some embodiments, methods are provided for treatment of pyrexia in equines, wherein pyrexia is due to a condition selected from viral, bacterial, and parasitic infections, respiratory infections, tick- and mosquito-borne infections, equine encephalomyelitis, equine influenza, equine herpes virus, West Nile virus, strangles, Potomac horse fever, colic, post-vaccination reactions, physical trauma and stress.

In some embodiments, methods are provided to treat pyrexia in an equine via a dosage regimen comprising administering metamizole to an equine in an amount in to the range of 20 to 50 mg metamizole per kg of equine subject weight, or in the range of 20 mg metamizole per kg of equine subject weight (mg/kg) to 40 mg/kg, or in the range of 20 mg metamizole per kg of equine subject weight (mg/kg) to 30 mg/kg, or in the range of 25 mg metamizole per kg of equine subject weight (mg/kg) to 35 mg/kg, or in the range of 25 mg metamizole per kg of equine subject weight (mg/kg) to 30 mg/kg, or in the range of 30 mg metamizole per kg of equine subject weight (mg/kg) to 35 mg/kg, or in the range of 27 mg metamizole per kg of equine subject weight (mg/kg) to 33 mg/kg, or in the range of 28 mg metamizole per kg of equine subject weight (mg/kg) to 32 mg/kg, or of 30 mg/kg of body weight, at a dosing interval of twice daily. In some embodiments, administration is intravenous. In some embodiments, administration is subcutaneous or intramuscular. In some embodiments, the metamizole is administered to the equine twice a day for at least 1 day, at least 2 days, at least 3 days, at least 4 days, at least 5 days, at least 6 days, at least 7 days, at least 8 days, or at least 9 days. In some embodiments, the metamizole is administered to the equine twice a day for 1 to 9 days, for 1 to 8 days, for 1 to 7 days, for 1 to 6 days, for 1 to 5 days, for 1 to 4 days, for 1 to 3 days, or for 1 to 2 days. In embodiments, the metamizole is administered no more than 18 times in a period of 9 days.

In some such embodiments, the metamizole is administered via intramuscular, intravenous (IV), or subcutaneous administration. In some embodiments, the metamizole is administered via intravenous administration. In some embodiments, the metamizole is administered via slow intravenous administration.

In some embodiments, the dose of metamizole is administered as a pharmaceutical formulation, i.e. the metamizole is comprised within a pharmaceutical formulation that may contain other ingredients. In some embodiments, the concentration of metamizole in the pharmaceutical formulation is from 200 mg/ml to 750 mg/ml or from 400 mg/ml to 600 mg/ml. In embodiments, the concentration of metamizole is 500 mg/ml.

In some embodiments, the metamizole is metamizole sodium monohydrate.

In some embodiments, the pyrexia is due to a condition selected from viral, bacterial, and parasitic infections, respiratory infections, tick- and mosquito-borne infections, equine encephalomyelitis, equine influenza, equine herpes virus, West Nile virus, strangles, Potomac horse fever, colic, post-vaccination reactions, physical trauma and stress.

### SUMMARY OF THE DRAWINGS

FIG. 1 is a flow diagram showing the design of the animal study as described in Example 1.

FIG. 2 is a flow diagram showing the design of the animal study as described in Example 2.

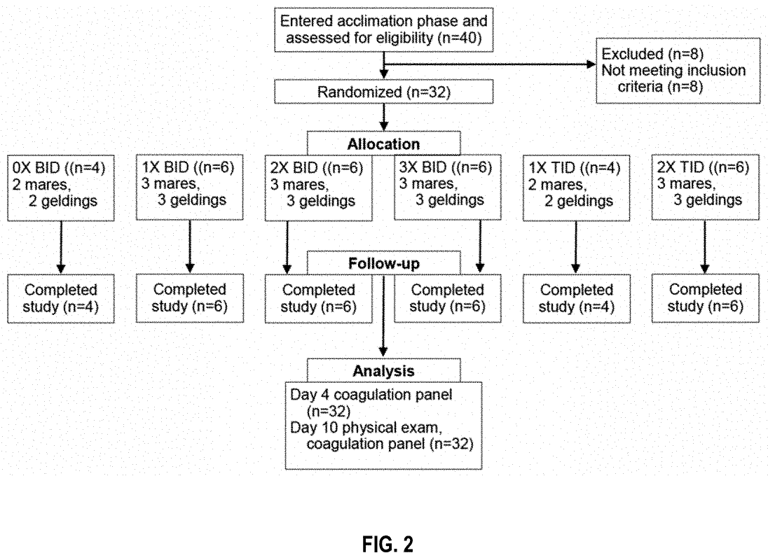

FIG. 3 demonstrates dose proportionality using data from the initial dose administered. The 4-MAA concentration for individual horse(s) at the 0.25 hour timepoint are shown. Treatment groups (30, 60 or 90 mg/kg) are organized by mg/kg dose. For example, the 1×TID and 1×BID dose groups are both included in the 30 mg/kg section.

FIG. 4 demonstrates the mean temperature over time (hours) relative to the first and crossover doses of metamizole (30 mg/kg) or placebo in horses with presumptive respiratory infections and a rectal temperature≥102.0° F. (see Study 2 of Example 3).

### DETAILED DESCRIPTION

In one aspect, the present disclosure provides methods of delivering therapeutically effective doses of metamizole to equines. To facilitate description of the invention, the following definitions are provided.

### Definitions

Unless defined otherwise below, all technical and scientific terms used herein have the same meaning as is commonly understood by one of ordinary skill in the art to which this disclosure pertains.

The terms “a” and “an” and “the” and similar referents as used herein refer to both the singular and the plural, unless otherwise indicated herein or clearly contradicted by context.

All numbers herein are approximate and should be treated to take into account measurement errors and rounding due to significant digits. Thus, for example, a reported dose of 30 mg/kg includes doses that range above or below 30 mg/kg by about 10%, due to errors or imprecision in measurement, and doses that are calculated to three significant figures but that would round up or down to 30 mg/kg, such as 29.6 mg/kg or 30.4 mg/kg.

The term “about” as used herein refers to greater or lesser than the value or range of values stated by 1/10 of the stated values but is not intended to limit any value or range of values to only this broader definition. For instance, a value of “about 30%” means a value of between 27% and 33%. Each value or range of values preceded by the term “about” is also intended to encompass the embodiment of the stated absolute value or range of values. Recitation of ranges of values herein are merely intended to serve as a shorthand method of referring individually to each separate value inclusively falling within the range, unless otherwise indicated herein, and each separate value is incorporated into the specification as if it were individually recited herein.

The term “metamizole” (also known as “dipyrone”) as used herein refers to [(2,3-dihydro-1,5-dimethyl-3-oxo-2-phenyl-1H-pyrazol-4-yl)methylamino] methanesulfonic acid and its pharmaceutically acceptable salts. Metamizole is available commercially (e.g., BOC Sciences) and generally has the structure shown in Formula I:

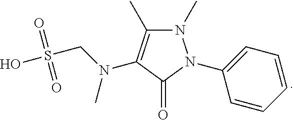 [SMILES: Cc1c(N(C)CS(=O)(=O)O)c(=O)n(-c2ccccc2)n1C]

Any reference to metamizole herein impliedly refers to any pharmaceutically acceptable salt (including ionized salts) and any related hydrated salt, polymorph, crystal, or pro-drug form thereof. Metamizole and metamizole sodium and metamizole sodium monohydrate are known compounds in the art and methods for their preparation may be found in the literature. An exemplary pharmaceutically acceptable salt of metamizole is the sodium salt having the structure in Formula II:

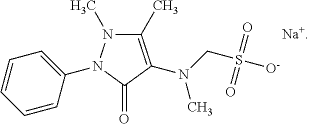 [SMILES: Cc1c(N(C)CS(=O)(=O)[O-])c(=O)n(-c2ccccc2)n1C]

Metamizole sodium monohydrate is metamizole sodium with a single water molecule associated with the metamizole sodium. In various embodiments provided herein, the stable active metabolite of metamizole, 4-methylaminoantipyrine (4-MAA), may be used in place of metamizole.

The phrase “pharmaceutical formulation” refers to a composition intended for therapeutic use that is safe and effective for its intended use. A formulation safe and effective for one type of use, such as IV administration, may not be safe or effective for another type of use, such as topical application. Thus, a “pharmaceutical formulation for IV administration”, for example, excludes any type of pharmaceutical formulation that would not be safe or effective for IV use. A pharmaceutical formulation of metamizole, for example, may contain other ingredients such as water or excipients.

The term “preservative” refers to an excipient suitable for use in a pharmaceutical formulation that is intended to help maintain the drug in a desired physical state. A preservative may have anti-microbial or anti-oxidant properties or may otherwise serve to protect the drug, e.g., from exposure to light or air. Suitable preservatives include parabens such as, without limitation, butyl, methyl or propyl paraben; sodium metabisulfate; potassium sorbate; sorbic acid; and butylated hydroxytoluene (BHT).

The term “pyrexia” refers to an elevation in body temperature (i.e., a fever) that is believed by a physician or veterinarian to require medical intervention to lower, e.g., to a safe set-point.

The phrase “therapeutically effective dose” refers to that amount of a drug (an “active pharmaceutical ingredient” or “API”) that is administered simultaneously or contemporaneously in one administration (multiple unit dose forms, i.e., pills, tablets, capsules, injections, intravenous, liquids, pastes can be administered in one administration) to achieve a desired therapeutic outcome, even if multiple administrations over time are administered in the course of therapy.

The phrase “slow intravenous administration” as used herein refers to an intravenous injection that is administered over a period of one to five minutes.

“Equine” as used herein is intended to be nonlimiting and refers to all types and breeds of horses, including all varieties of ponies (e.g. Shetland, Welsh, German riding pony, etc.), all varieties of horses (e.g. Arabians, thoroughbreds, standardbreds, warmbloods, quarter horses, appaloosas, mustangs, and the like), draft horses (e.g. Clydesdales, Belgians, Friesians, and the like), and miniature horses. Equines also include related species such as mules, donkeys, and zebras.

As used herein, “treatment” refers to therapeutic intervention, for example, wherein the object is to reduce the severity of the targeted pathologic condition or disorder or improve at least one symptom of the disorder, for example, by reducing a fever in an animal. Treatment also refers to, for example, therapeutic intervention wherein the object is to prevent or inhibit onset of a condition or disorder such as pyrexia after exposure or suspected exposure to a causative agent such as a colic or a viral or bacterial infection.

### Exemplary Methods

In one aspect, the present disclosure provides a method of treating pyrexia in an equine comprising administering a therapeutically effective dose of metamizole to the equine patient. In some embodiments, the methods provided herein include repeat daily administration, including, without limitation, up to 2 times daily. In some embodiments, administration is repeated no more than 2 times daily or no more than every 12 hours. In some embodiments, the metamizole is administered to the equine twice a day for at least 1 day, at least 2 days, at least 3 days, at least 4 days, at least 5 days, at least 6 days, at least 7 days, at least 8 days, or at least 9 days. In some embodiments, the metamizole is administered to the equine twice a day for 1 to 9 days, 1 to 8 days, for 1 to 7 days, for 1 to 6 days, for 1 to 5 days, for 1 to 4 days, for 1 to 3 days, or for 1 to 2 days. In some embodiments, treatment is given for no more than 9 days, e.g. no more than 18 doses in 9 days. In various embodiments of these methods, the therapeutically effective dose is in the range of 20 mg metamizole per kg of equine subject weight (mg/kg) to 50 mg/kg, or in the range of 20 mg metamizole per kg of equine subject weight (mg/kg) to 40 mg/kg, or in the range of 20 mg metamizole per kg of equine subject weight (mg/kg) to 30 mg/kg, or in the range of 25 mg metamizole per kg of equine subject weight (mg/kg) to 35 mg/kg, or in the range of 25 mg metamizole per kg of equine subject weight (mg/kg) to 30 mg/kg, or in the range of 30 mg metamizole per kg of equine subject weight (mg/kg) to 35 mg/kg, or in the range of 27 mg metamizole per kg of equine subject weight (mg/kg) to 33 mg/kg, or in the range of 28 mg metamizole per kg of equine subject weight (mg/kg) to 32 mg/kg. In an embodiment, the dose is 30 mg metamizole per kg of equine subject weight (mg/kg).

In embodiments, the unit dose is administered in a formulation in which metamizole is present at a w/w percentage of at least 25% and typically at least 50% of the formulation, enabling a unit dose to be in the range of 20-50 ml in an adult horse. In some embodiments, the concentration of metamizole in the pharmaceutical formulation is from 200 mg/ml to 750 mg/ml or from 400 mg/ml to 600 mg/ml. In embodiments, the concentration of metamizole is 500 mg/ml.

In various embodiments, a therapeutically effective dose of metamizole for practice of the methods provided herein is in the range of about 20 mg to 50 mg metamizole/per kg weight of animal (mg/kg)/per dose. In some embodiments, metamizole is administered at a dosage of about 30 mg/kg. For example, in some embodiments, the dose is in the range of 20 mg metamizole per kg of equine subject weight (mg/kg) to 50 mg/kg, or in the range of 20 mg metamizole per kg of equine subject weight (mg/kg) to 40 mg/kg, or in the range of 20 mg metamizole per kg of equine subject weight (mg/kg) to 30 mg/kg, or in the range of 25 mg metamizole per kg of equine subject weight (mg/kg) to 35 mg/kg, or in the range of 25 mg metamizole per kg of equine subject weight (mg/kg) to 30 mg/kg, or in the range of 30 mg metamizole per kg of equine subject weight (mg/kg) to 35 mg/kg, or in the range of 27 mg metamizole per kg of equine subject weight (mg/kg) to 33 mg/kg, or in the range of 28 mg metamizole per kg of equine subject weight (mg/kg) to 32 mg/kg. In an embodiment, the dose is 30 mg metamizole per kg of equine subject weight (mg/kg).

Typically, the therapeutically effective dose is administered no more than twice daily, and daily administration continues for several days or longer, but single day treatments can be effective in some animals for some purposes. Generally, however, treatment will continue on consecutive days for several days to a week, or longer. The dose may be adjusted using kg of weight of the animal to be treated. In some embodiments, administration with a metamizole formulation provided herein is continued for 2, 3, 4, 5, 6, 7, 8, 9, or more consecutive days. In some embodiments, administration is repeated no more than 2 times daily or no more than every 12 hours. In some embodiments, the metamizole is administered to the equine twice a day for at least 1 day, at least 2 days, at least 3 days, at least 4 days, at least 5 days, at least 6 days, at least 7 days, at least 8 days, or at least 9 days. In some embodiments, the metamizole is administered to the equine twice a day for 1 to 9 days, for 1 to 8 days, for 1 to 7 days, for 1 to 6 days, for 1 to 5 days, for 1 to 4 days, for 1 to 3 days, or for 1 to 2 days. In some embodiments, treatment is given for no more than 9 days, e.g. no more than 18 doses in 9 days.

In various embodiments, methods are provided wherein the metamizole is administered via intramuscular, intravenous (IV), or subcutaneous administration. In some embodiments, the metamizole is administered via intravenous administration. In some embodiments, the metamizole is administered via slow intravenous administration. In some embodiments, the metamizole is metamizole sodium monohydrate.

In some embodiments, methods of treating pyrexia in an equine using a dosage regimen are provided. In some embodiments, the dosage regimen comprises administering metamizole parenterally to an equine at a dosing interval of twice daily. In embodiments, the metamizole is administered intravenously. In some embodiments, metamizole may be administered for 2, 3, 4, 5, 6, 7, 8, 9, or more consecutive days. In some embodiments, administration is repeated no more than 2 times daily or no more than every 12 hours. In some embodiments, the metamizole is administered to the equine twice a day for at least 1 day, at least 2 days, at least 3 days, at least 4 days, at least 5 days, at least 6 days, at least 7 days, at least 8 days, or at least 9 days. In some embodiments, the metamizole is administered to the equine twice a day for 1 to 9 days, for 1 to 8 days, for 1 to 7 days, for 1 to 6 days, for 1 to 5 days, for 1 to 4 days, for 1 to 3 days, or for 1 to 2 days. In some embodiments, treatment is given for no more than 9 days, e.g. no more than 18 doses in 9 days. The dosage regimen may be continued until the equine no longer requires treatment with metamizole. In some embodiments, metamizole is administered in an amount of 30 mg/kg of body weight of the equine. For example, in some embodiments, the dose is in the range of 20 mg metamizole per kg of equine subject weight (mg/kg) to 50 mg/kg, or in the range of 20 mg metamizole per kg of equine subject weight (mg/kg) to 40 mg/kg, or in the range of 20 mg metamizole per kg of equine subject weight (mg/kg) to 30 mg/kg, or in the range of 25 mg metamizole per kg of equine subject weight (mg/kg) to 35 mg/kg, or in the range of 25 mg metamizole per kg of equine subject weight (mg/kg) to 30 mg/kg, or in the range of 30 mg metamizole per kg of equine subject weight (mg/kg) to 35 mg/kg, or in the range of 27 mg metamizole per kg of equine subject weight (mg/kg) to 33 mg/kg or in the range of 28 mg metamizole per kg of equine subject weight (mg/kg) to 32 mg/kg. In an embodiment, the dose is 30 mg metamizole per kg of equine subject weight (mg/kg).

In some embodiments, the metamizole is metamizole sodium monohydrate.

In some embodiments, the present disclosure provides a pharmaceutical formulation comprising metamizole that may be administered to equines. In embodiments, the pharmaceutical formulation is suitable for parenteral administration, including, without limitation, intramuscular, intravenous (IV), and subcutaneous administration. In embodiments, the metamizole is metamizole sodium monohydrate.

Typically, the therapeutically effective amount for an adult equine will be a unit dose of the therapeutic formulation sufficient to administer a dose of metamizole in the range of 10-60 mg of metamizole per kg of subject weight, i.e., typically about 25-50 mg/kg or 30-40 mg/kg, and so for the typical 350 kg to 1000 kg adult mare, a typical therapeutically effective (single) dose administered at, e.g., 30 mg/kg would be in the range 10.5 g to 30 g. For a foal, the therapeutically effective amount will be a unit dose of the therapeutic formulation sufficient to administer a dose of metamizole in the range of 10-50 mg of metamizole per kg of subject weight, i.e., typically about 15-40 mg/kg, and so for the typical foal (for example, weighing approximately 35-100 kg), a typical therapeutically effective (single) dose administered at, e.g., 20 mg/kg would be in the range of 700 to 2000 mg.

In some embodiments of the present disclosure, a pharmaceutical composition contains metamizole at a concentration sufficient to enable dosing at about 20-50 mg/kg, i.e., concentrations in the range of about 200 mg/ml to 750 mg/ml, or 250 mg/ml to 750 mg/ml. Typically, the administered dose will be in the range of from 10 to 60 mg/kg, 10 to 50 mg/kg, or 10 to 40 mg/kg, or 20 to 50 mg/kg, or 20 to 40 mg/kg, or 10 mg/kg to 35 mg/kg, or 10 to 30 mg/kg, or 15 to 35 mg/kg, or 15 to 30 mg/kg, or 20 to 30 mg/kg, or 25 to 35 mg/kg, or 30 to 35 mg/kg, or 27-33 mg/kg, or 28-32 mg/kg, or 30 mg/kg. In some embodiments of the present disclosure the pharmaceutical composition contains metamizole in a range of about 25% w/w to 50% w/w.

In some embodiments, the therapeutically effective dose is 30 mg/kg. In embodiments, the concentration of metamizole in the formulation is about 500 mg/ml.

In some embodiments, the therapeutically effective unit dose has a volume of from about 10 to 100 ml, or about 10 to 75 ml, or about 10 to 50 ml, and preferably of about 20 ml to 30 ml.

The pharmaceutical formulation may further include one or more additional excipients. For example, in some embodiments the pharmaceutical formulation contains one or more preservative agents. In some embodiments, the pharmaceutical formulation contains one or more preservative agents selected from the group consisting of alcohol, benzyl alcohol, bronopol, chlorbutol, chlorocreson, a paraben such as, without limitation, butyl, methyl or propyl paraben, phenol, phenylethanol, sodium benzoate, potassium sorbate, sorbic acid, glycerin, and propylene glycol. In some embodiments, the pharmaceutical composition contains a preservative agent in a range of 0.05% w/w to 0.5% w/w, typically the amount is about 0.2% w/w. In some embodiments, the pharmaceutical composition contains at least two preservative agents such as methylparaben and propylparaben. In other embodiments, the pharmaceutical compositions contain a preservative that is an antioxidant, such as sodium metabisulfite. In embodiments, various formulations include sodium metabisulfite, methylparaben, and propylparaben.

The pharmaceutical formulation provided herein may further comprise a buffering agent, such as a buffering salt and/or a pH buffering additive. In some embodiments, the pharmaceutical composition contains a preservative agent with solvent properties, such as propylene glycol, at a higher concentration, for example, at a concentration of 10 mg/ml to 500 mg/ml, or 10 mg/ml to 300 mg/ml, or 50 mg/ml to 300 mg/ml, or 50 mg/ml to 200 mg/ml, or 50 mg/ml to 150 mg/ml.

### EXAMPLES

### Example 1: Evaluation of the Safety of Metamizole Administered Intravenously Three Times Daily in Horses

A study was conducted to evaluate the margin of safety of metamizole when administered intravenously to horses for 9 days at doses of 0 mg/kg (0×), 30 mg/kg (1×), 60 mg/kg (2×), and 90 mg/kg (3×) of body weight three times daily (TID), every 8 hours±30 min.

### Study Design

This was a prospective, blinded, randomized, placebo-controlled, parallel-group laboratory study in research horses. Thirty-two horses were enrolled in the study (Table 1).

TABLE 1
Treatment Groups

| | | | | | Total Dose | |
|---|---|---|---|---|---|---|
| | | Number | IV Dosage | | per Day | Dose |
| Group | Treatment | (Male/Female) | (mg/kg) | Dose Frequency | (mg/kg) | Level |
| 1 | 0.9% NaCl | 4/4 | 0 | Every 8 | 0 | 0X |
| | | | | hours | | |
| 2 | Dipyrone | 4/4 | 30 | Every 8 | 90 | 1X |
| | | | | hours | | |
| 3 | Dipyrone | 4/4 | 60 | Every 8 | 180 | 2X |
| | | | | hours | | |
| 4 | Dipyrone | 4/4 | 90 | Every 8 | 270 | 3X |
| | | | | hours | | |

IV = intravenous

### Horses and Acclimation

Horses were purchased from a variety of sources, including auctions, and assembled in a herd at the research laboratory. Forty-one adult horses were screened for participation in the study. Inclusion criteria included: 3 to 20 years of age, 350 to 650 kg body weight, bilateral patent jugular veins, amenable to handling and study procedures, and no known history of medication use within 14 days of study start. Exclusion criteria included pregnancy, lactation, ill thrift, known history of NSAID sensitivity, and grade 4 gastric ulceration based on the Equine Gastric Ulcer Council scoring system of grades 0-4. (Sykes, Hewetson et al., 2015) Horses had gastroscopy performed pre-acclimation to determine baseline scoring. All horses were treated with omeprazole (Gastrogard®, Merial Limited, Duluth, Ga.) (4 mg/kg, PO, q24 hours) during acclimation (range 8-11 days). Omeprazole treatment was stopped two days prior to first dosing in all horses regardless of score and repeat gastroscopy was performed.

Horses were housed in individual pens at the research laboratory within a covered barn. Individual pens allowed contact with neighboring horses. Diet consisted of hay fed two to three times daily, a concentrate fed once daily, and water ad libitum. Horses were given unique identification collars and microchipped. Pasture turnout was not permitted due to the presence of indwelling intravenous catheters required for dosing. Horses were acclimated to study conditions in their individual pens for 2 weeks prior to initiation of the treatment phase (dosing period).

### Blinding and Randomization

The study director, study veterinarians, and study site personnel involved in data collection and observations were blinded to treatment allocation throughout the study. Dose administrators and the statistician were unblinded; however, they did not participate in data collection.

A multiple-stage randomization scheme was used to balance the 32 horses for sex, pen location, and necropsy cohort. First, horses were randomly assigned to 4 barn floor blocks (each barn floor block formed a necropsy cohort) such that each block consisted of eight horses (4 mares and 4 geldings). Then, within each barn floor block and sex, 4 horses were randomly assigned to 4 pens. Lastly, within each barn floor block and sex, 4 horses were randomly assigned to 4 treatment groups.

### Treatment Phase (Investigational Drug and Placebo)

Treatment phase involved administration of the investigational drug or placebo. The investigational drug was a sterile injectable formulation of dipyrone (Zimeta™, Corden Pharma, Caponago, Italy) (500 mg/mL) in 100 mL glass bottles. The placebo was sterile injectable saline (Nova-Tech Inc, Grand Island, NE) (0.9% sodium chloride) in 500 mL plastic bottles labeled for veterinary use. Dipyrone treatment groups were 30 mg/kg (1×), 60 mg/kg (2×), and 90 mg/kg (3×). Placebo treatment group was saline administered at a volume to match the 3× dipyrone group (0×). The dose was calculated based on body weight and administered three times daily for nine consecutive days. Due to the large volume of injection at the higher dose groups, treatment was administered via indwelling intravenous (IV) catheters. Intravenous catheters were placed using standard techniques. Horses were sedated with xylazine (AnaSed® LA, VetOne, MWI, Boise, Id.) and the skin was desensitized with subcutaneous injection of lidocaine (VetOne, MWI, Boise, Id.). Intravenous catheters were replaced at the discretion of the study director and were maintained with heparinized saline flush prior to and following dose administration.

### Schedule of Events

During acclimation and prior to inclusion all horses underwent a physical examination, nasal lavage for culture to rule out Streptococcus equi subsp. equi (S. equi), gastroscopy, blood collection for clinical pathology (hematology, serum chemistry, coagulation panel), and urine collection for urinalysis. Randomization occurred on Study Day −1. On Study Day −1 (baseline), Day 4, and Day 8 horses underwent a physical exam and blood collection for clinical pathology. Horses were dosed according to randomization beginning on Day 1 and were observed for two hours following each dose administration for adverse events. On Day 9, horses were sedated with detomidine (Dormosedan®, Zoetis, Kalamazoo, Mich.) (10-15 mg/horse, IV) and euthanized with sodium pentobarbital (Beuthanasia D Special, Intervet Inc, Madison, NJ) (46.8 g/horse, IV). A gross necropsy was completed and select tissues were collected for histopathological examination. In addition, due to the unexpected occurrence of pneumonia in the study population, lung samples were collected at necropsy and submitted for bacterial culture (UC Davis RT-PCR Research and Diagnostics Core Facility, Davis, CA) on horses selected by the pathologist that had gross signs of pneumonia. Urine collection for urinalysis was completed at necropsy for all horses.

TABLE 2
Necropsy Tissues Collected and Evaluated by Histopathology

| | Organ | |
|---|---|---|
| Necropsy Tissue | weighed | Histopathology |
| Adrenal glands | | X |
| Bone with marrow (sternum) | | X |
| Bone marrow smear (sternum) | | X |
| Brain (cerebrum, midbrain, cerebellum, | X | X |
| medulla oblongata) | | |
| Eyes with optic nerve | | X |
| Heart (right and left atriums, right and left | X | X |
| AV valves, pulmonary artery with pulmonic | | |
| valve, right and left ventricles, ventricular | | |
| septum, papillary muscle, and aorta with | | |
| aortic valve). | | |
| Kidneys | X | X |
| Jugular vein, catheter location | | X |
| Large intestine, cecum | | X |
| Large intestine, colon | | X |
| Liver, right & left lobes | X | X |
| Lung, peripheral and sample with | | X |
| large bronchus | | |
| Lymph node: submandibular, | | X |
| mediastinal, and mesenteric | | |
| Mammary gland (if applicable) | | X |
| Ovaries (if applicable) | X | X |
| Pancreas | | X |
| Pituitary | | X |
| Skeletal muscle | | X |
| (semimembranosus/semitendinosus) | | |
| Skin | | X |
| Small intestine, duodenum | | X |
| Small intestine, ileum | | X |
| Small intestine, jejunum | | X |
| Spinal cord: cervical, thoracic, lumbar | | X |
| Spleen | X | X |
| Stomach, glandular | | X |
| Stomach, nonglandular | | X |
| Thymus | | X |
| Thyroid glands | | X |
| Urinary bladder | | X |
| Uterus (if applicable) | | X |
| Gross Lesions (if applicable) | | X |
| Sample for diagnostic microbiology | | X |
| (if applicable based on gross findings) | | |

### Statistics

All statistical analyses were performed using SAS, version 9.4 (SAS Institute, Cary, N.C.). The individual horse was the experimental unit in the statistical analysis. For statistical modeling, all main effects, interaction terms, and pairwise comparisons of the active groups (dipyrone) with the placebo group were assessed at the two-sided 0.10 significance level (unadjusted) with the exception of the three-way interaction, which was assessed at the two-sided 0.05 significance level (unadjusted).

For continuous endpoints with single post-treatment measures (organ weight and organ weight relative to body weight), analysis of variance (ANOVA) was used to evaluate a model including treatment, sex, and treatment by sex interaction as fixed effects. If treatment by sex interaction was significant, within sex pair-wise comparison of each treatment group versus placebo using linear contrasts were performed. If treatment by sex interaction was not significant and treatment effect was significant, pair-wise comparison of each treatment group versus placebo for pooled sex were performed. In particular, sex specific organ weights (ovaries) were analyzed by ANOVA including only treatment as a fixed effect.

For continuous endpoints with repeated post-treatment measurements (serum chemistry, coagulation, hematology, urinalysis, body weight, body temperature, heart rate and respiratory rate), repeated measurement analysis of covariance (SAS MIXED procedure RMANCOVA) was used to evaluate a model including treatment, time, sex, treatment by sex interaction, treatment by time interaction, and treatment by time by sex three-way interaction as fixed effects. In addition, baseline values were included as a covariate and remained in the model regardless of its significance. Baseline values were defined as the values prior to and nearest to the first dosing. The covariance structures for repeated measures analysis of covariance (RMANCOVA) were selected from compound symmetry (CS) and compound symmetry heterogeneous variances (CSH), since only two post baseline measurements were collected in this study. The Kenward-Roger approximation was used to calculate denominator degrees of freedom for statistical tests. The minimum Akaike Information Criterion was used to select the covariance structure.

If the 3-way interaction (treatment by time by sex) was significant then the statistical analysis was considered inconclusive and no further statistical analyses were conducted. If the 3-way interaction was not significant then treatment by time and treatment by sex interactions were evaluated. If the treatment by time interaction was significant (regardless of significance of treatment by sex interaction), within time pair-wise comparisons of each treatment group versus placebo using linear contrasts were performed. If treatment by sex interaction was significant (regardless of significance of treatment by time interaction), within sex pair-wise comparisons of each treatment group versus placebo using linear contrasts were performed.

### Adverse Events

All 32 horses were acclimated prior to study initiation and started the treatment phase healthy. During the study, numerous horses in all groups including placebo developed clinical signs of pneumonia likely due to exposure and incubation of respiratory pathogens prior to the start of the treatment phase. The mean time to development of pneumonia was 6.9 days with a range of 4.3 to 8.3 days. One horse in the 3× group died due to severe pneumonia on Day 8. The other 31 horses completed the study. At the second gastroscopy examination, completed just prior to the treatment phase, one horse was noted to have purulent material draining from the guttural pouch. The endoscope was appropriately cleaned before completing the remaining gastroscopies and that horse was not randomized to the study; however, she had been housed, and in contact, with the enrolled horses and is considered suspect for the initial spread of infection.

### Concomitant Medications

Due to the severity of pneumonia, two horses received concomitant treatments. The most severely affected horse was treated with furosemide 5% (Bayer HealthCare LLC, Shawnee Mission, KS) (1 mg/kg, IV, q6 hours), ceftiofur (Naxcel®, Zoetis, Pharmacia & Upjohn Company, NY, NY) (2.2 mg/kg, IM, q 12 hours), and Vitamin K (Vitamin K 1, VetOne, MWI, Boise, Id.) (0.6 mg/kg, SQ, q 4 hr). A second horse was treated with ceftiofur. Two additional horses received other concomitant medications during the acclimation period for unrelated medical needs including treatment of a leg wound (sedation and antibiotics) and sedation for radiographs to assess foot lameness.

### Statistical Results

There was a statistically significant main treatment effect of prolongation of prothrombin time (PT) with dipyrone treatment. There were statistically significant treatment by sex interactions for percent basophils and absolute basophil count. There were statistically significant treatment by time interactions for cholesterol, creatinine, total bilirubin, mean corpuscular volume (MCV), and mean corpuscular hemoglobin concentration in the dipyrone groups compared to placebo (Table 3).

There was a statistically significant increase in absolute and relative liver weights. No significant differences were found for urinalysis, body weights, and physical examination parameters. There were dose dependent increases in prothrombin time, total bilirubin and liver weights apparent in the dipyrone treated (2× and 3×) groups compared to placebo. Renal, hepatic and gastrointestinal gross necropsy and histopathology evaluation showed no significant findings even at higher doses administered every 8 hours. Table 4 summarizes the statistically significant findings at specific time points following initiation of dosing. Table 5 summarizes the range of clinical pathology values for those parameters with a statistically significant finding.

TABLE 3
Summary of the Repeated Measures of ANCOVA

| | P-value | | | | | |
|---|---|---|---|---|---|---|
| | | | | | Treatment * | |
| | | | Treatment * | Treatment * | Sex * | |
| Parameter | Baseline | Treatment | Sex | Time | Time | Decisions |
| Cholesterol | <0.001 | 0.019 | 0.759 | 0.088 | 0.540 | 2 |
| Creatinine | 0.006 | 0.814 | 0.916 | 0.020 | 0.113 | 2 |
| Total Bilirubin | 0.022 | 0.001 | 0.724 | 0.067 | 0.054 | 2 |
| Prothrombin | 0.035 | 0.004 | 0.439 | 0.694 | 0.671 | 4 |
| Time | | | | | | |
| Percent Basophil | 0.241 | 0.204 | 0.050 | 0.491 | 0.220 | 3 |
| Absolute Basophil | 0.884 | 0.304 | 0.077 | 0.567 | 0.179 | 3 |
| Count | | | | | | |
| Mean Corpuscular | <0.001 | 0.020 | 0.864 | 0.011 | 0.854 | 2 |
| Volume | | | | | | |
| Mean Corpuscular | 0.329 | 0.074 | 0.570 | 0.100 | 0.711 | 2 |
| Hemoglobin | | | | | | |
| Concentration | | | | | | |

Decisions:
2 = The treatment * time interaction is significant at 0.1. Follow up treatment mean comparisons will be made within time;
3 = The treatment * sex interaction is significant at 0.1. Follow up treatment mean comparisons will be made within sex.;
4 = Neither the treatment * time nor the treatment * sex interaction is significant at 0.1 but the main effect of treatment is significant. Follow up treatment mean comparisons will be made averaged over time and sex.;
P-values in bold-face indicate significant fixed effects.

TABLE 4
Clinical Pathology and Necropsy Findings

| Parameter | Timepoint | Treatment | LS Means | SEM | P-value |
|---|---|---|---|---|---|
| Cholesterol | Day 4 | 0X | 67.527 | 2.6419 | n/a |
| (mg/dL) | | 1X | 65.352 | 2.6257 | 0.565 |
| | | 2X | 56.772 | 2.6834 | 0.006 |
| | | 3X | 54.144 | 2.7179 | 0.001 |
| Creatinine | Day 8 | 0X | 1.158 | 0.0803 | n/a |
| (mg/dL) | | 1X | 1.118 | 0.0805 | 0.727 |
| | | 2X | 0.958 | 0.0803 | 0.087 |
| | | 3X | 1.144 | 0.0852 | 0.902 |
| Total Bilirubin | Day 4 | 0X | 1.505 | 0.3531 | n/a |
| (mg/dL) | | 1X | 2.301 | 0.3428 | 0.121 |
| | | 2X | 2.745 | 0.3438 | 0.019 |
| | | 3X | 3.074 | 0.3404 | 0.003 |
| | Day 8 | 0X | 2.405 | 0.3531 | n/a |
| | | 1X | 2.914 | 0.3428 | 0.317 |
| | | 2X | 4.495 | 0.3438 | <0.001 |
| | | 3X | 4.579 | 0.3602 | <0.001 |
| Prothrombin | n/a | 0X | 12.734 | 0.4041 | n/a |
| Time | | 1X | 13.374 | 0.4062 | 0.276 |
| (sec) | | 2X | 14.524 | 0.4064 | 0.005 |
| | | 3X | 14.946 | 0.4396 | 0.001 |
| Mean | Day 4 | 0X | 46.047 | 0.1782 | n/a |
| Corpuscular | | 1X | 46.512 | 0.1785 | 0.079 |
| Volume (fL) | | 2X | 46.284 | 0.1779 | 0.358 |
| | | 3X | 46.560 | 0.1781 | 0.053 |
| | Day 8 | 0X | 46.384 | 0.2269 | n/a |
| | | 1X | 47.000 | 0.2271 | 0.069 |
| | | 2X | 47.134 | 0.2266 | 0.029 |
| | | 3X | 47.708 | 0.2370 | <0.001 |
| Mean | Day 8 | 0X | 35.232 | 0.4700 | n/a |
| Corpuscular | | 1X | 35.513 | 0.4700 | 0.677 |
| Hemoglobin | | 2X | 34.080 | 0.4704 | 0.096 |
| Concentration | | 3X | 33.899 | 0.5078 | 0.066 |
| (g/dL) | | | | | |
| Percent | Mares | 0X | 0.885 | 0.2772 | n/a |
| Basophil (%) | | 1X | 1.374 | 0.2764 | 0.228 |
| | | 2X | 0.520 | 0.2766 | 0.362 |
| | | 3X | 1.731 | 0.3156 | 0.057 |
| | Geldings | 0X | 1.558 | 0.3012 | n/a |
| | | 1X | 0.849 | 0.2778 | 0.093 |
| | | 2X | 0.949 | 0.2781 | 0.163 |
| | | 3X | 0.924 | 0.2812 | 0.154 |
| Absolute | Mares | 0X | 0.102 | 0.0370 | n/a |
| Basophil Count | | 1X | 0.200 | 0.0370 | 0.076 |
| (cells 10³/μL) | | 2X | 0.059 | 0.0371 | 0.412 |
| | | 3X | 0.188 | 0.0430 | 0.147 |
| Liver (absolute | n/a | 0X | 5808.13 | 434.513 | n/a |
| weight) (g) | | 1X | 6280.00 | 434.513 | 0.450 |
| | | 2X | 6892.50 | 434.513 | 0.091 |
| | | 3X | 7765.21 | 469.327 | 0.006 |
| Liver (relative | n/a | 0X | 12.29 | 0.912 | n/a |
| to body weight) | | 1X | 13.69 | 0.912 | 0.290 |
| | | 2X | 15.28 | 0.912 | 0.030 |
| | | 3X | 16.38 | 0.985 | 0.006 |

0X, placebo;
1X, 30 mg/kg dipyrone;
2X, 60 mg/kg dipyrone;
3X, 90 mg/kg dipyrone;
LS means, Least Square means;
SEM, standard error of the mean;
n/a, non-applicable.
Bolded p-values are statistically significant at P < 0.1.

TABLE 5
Reference Range Evaluation of Statistically Significant Clinical Pathology Parameters

| | Treatment | Baseline | Day 4 | Day 8 | Reference |
|---|---|---|---|---|---|
| Parameter | Group | Range | Range | Range | range |
| Cholesterol (mg/dL) | 0X | 51-92 | 48-78 | 57-86 | 50-140 |
| | 1X | 53-92 | 54-77 | 64-87 | |
| | 2X | 58-79 | 43-74 | 52-87 | |
| | 3X | 56-108 | 43-73 | 55-96 | |
| Creatinine (mg/dL) | 0X | 1.2-1.8 | 1.2-1.7 | 0.8-1.4 | 1-2.2 |
| | 1X | 1.2-1.8 | 0.9-2.0 | 0.6-1.6 | |
| | 2X | 1.2-1.7 | 1.1-2.0 | 0.6-1.4 | |
| | 3X | 1.1-1.8 | 1.1-2.1 | 0.7-1.6 | |
| Total Bilirubin (mg/dL) | 0X | 0.9-2.8 | 0.9-2.6 | 1.2-4.2 | 0.8-3.2 |
| | 1X | 0.6-2.4 | 0.8-3.4 | 1.7-4.1 | |
| | 2X | 0.8-2.2 | 1.6-4.0 | 1.6-6.1 | |
| | 3X | 0.9-3.0 | 1.7-5.5 | 2.8-6.2 | |
| Prothrombin Time (sec) | 0X | 12.3-13.4 | 12.1-13.2 | 11.4-14.7 | 9-12 |
| | 1X | 12.2-13.7 | 12.6-14.6 | 12.3-15.2 | |
| | 2X | 12.0-13.4 | 12.7-15.8 | 12.8-21.6 | |
| | 3X | 12.5-14.0 | 13.4-15.9 | 12.7-21.9 | |
| Percent Basophil (%) | 0X | 0.7-7.9 | 0.0-1.8 | 0.0-4.0 | 0-0.1 |
| | 1X | 0.6-5.7 | 0.0-2.3 | 0.0-2.6 | |
| | 2X | 0.4-4.3 | 0.0-1.3 | 0.0-2.7 | |
| | 3X | 0.4-3.8 | 0.0-4.0 | 0.0-2.1 | |
| Absolute Basophil Count (cells | 0X | 0.07-0.62 | 0.00-0.16 | 0.00-0.47 | 0-0.7 |
| 10³/μL) | 1X | 0.08-0.43 | 0.00-0.19 | 0.00-0.52 | |
| | 2X | 0.05-0.46 | 0.00-0.12 | 0.00-0.21 | |
| | 3X | 0.04-0.56 | 0.00-0.39 | 0.00-0.41 | |
| Mean Corpuscular Hemoglobin | 0X | 33.5-35.8 | 34.4-35.9 | 34.1-39.4 | 36.5-39.3 |
| Concentration (g/dL) | 1X | 27.9-35.6 | 34.9-36.1 | 34.1-39.4 | |
| | 2X | 33.1-35.6 | 34.2-36.1 | 32.9-35.5 | |
| | 3X | 27.8-35.7 | 34.3-36.0 | 33.1-34.5 | |
| Mean Corpuscular Volume (fL) | 0X | 43.2-50.3 | 43.0-49.2 | 42.8-49.9 | 37-51.4 |
| | 1X | 41.1-49.2 | 40.8-48.7 | 40.8-49.3 | |
| | 2X | 43.3-49.6 | 41.7-49.4 | 43.2-50.8 | |
| | 3X | 43.6-50.5 | 43.0-50.4 | 43.8-51.3 | |

Underlined values are below the reference range.
Bolded values are above the reference range.

### Coagulation Parameters

Almost all individual horses were above the PT laboratory reference range of 9 to 12 seconds on Days −1, 4, and 8 (Table 5). A wide range of reference ranges are used in equine medicine due to differences in laboratory methodologies, reagents, and procedures for generation of reference ranges. There was a statistically significant main treatment effect for PT in the 2× and 3× dose groups representing a dose related prolongation in PT. There were no statistically significant differences in activated partial thromboplastin time (APTT) or fibrinogen. The statistically significant prolongation of PT was mildly dose-dependent at multiples of the proposed dose. Prolongation of PT was observed in two horses (2× group n=1, 3× group n=1) on Day 8. Prothrombin time measures the activities of the extrinsic and common coagulation pathway factors.

In Table 6 below the percent of samples in each treatment group that were above the upper reference range limit of 12 seconds is listed for Days 4 and 8. The percent of samples in each treatment group that were greater than 14 secs is listed along with the range of these elevated samples. The cutoff of 14 seconds was chosen to eliminate the vast majority of samples which were slightly above the reference range of 12 secs.

TABLE 6
Elevation in Prothrombin Time Values Per Treatment Group

| | | Percent of Elevated PT Values per Treatment Group | | | | |
|---|---|---|---|---|---|---|
| | | Day 4 | | Day 8 | | |
| Dose Level | PT (sec)¹ | % >12 sec | % >14 sec (range sec) | % >12 sec | % >14 sec (range sec) | |
| 0X | 12.734 ± 0.4041 | 100%² | 0%³ | 75%² | 13%³ | (14.7) |
| 1X | 13.374 ± 0.4062 | 100% | 25% (14.1-14.6) | 100% | 13% | (15.2) |
| 2X | 14.524 ± 0.4064* | 100% | 13% (15.8) | 100% | 50% | (14.7-21.6) |
| 3X | 14.946 ± 0.4396* | 100% | 75% (14.6-15.9) | 100%⁴ | 71%⁴ | (14.3-21.9) |

¹LSmean ± SEM.
²Percent of samples per treatment group with PT >12 sec (ref. range upper limit).
³Percent of
⁴On Day 8 only 7 samples were analyzed in group 3X due to prior death of Animal 161.
*Statistically significant main treatment effect versus controls (P ≤ 0.10). Reference range is 9 to 12 sec.

Prolonged PT can be caused by numerous genetic abnormalities and platelet dysfunction; however, few abnormalities result in an increase in PT in a short time period. Platelet dysfunction may be due to a variety of causes including liver disease, uremia, severe anemia, drug or toxin-induced dysfunction, and disseminated intravascular coagulation. Cox-1 inhibition can result in platelet dysfunction but is reversible and has not been reported to impact PT parameters in the horse. Both horses with specific prolongation were found to have lung abscessation and cultured positive for Staphylococcus aureus (S. aureus) and Streptococcus equi subsp zooepidemicus (S. zooepidemicus).

TABLE 7
Lung Culture Results

| | Treatment Group (n = number positive |
|---|---|
| Organism cultured at necropsy | horses) |
| S. aureus | 1X (n = 1), 2X (n = 2), 3X (n = 2) |
| S. equi subsp. zooepidemicus | 0X (n = 2), 2X (n = 1), 3X (n = 1) |
| S. dysgalactiae | 2X (n = 1) |
| subsp. equisimilis | |
| Aeromonas sp. | 2X (n = 1) |
| Acinetobacter sp. | 2X (n = 1) |
| Pseudomonas sp. | 2X (n = 1) |
| Pantoea sp. | 2X (n = 1) |

Positive lung culture results were obtained at necropsy from 9 horses (0X n = 2, 1X n = 1, 2X n = 4, 3X n = 2). Samples were positive for more than one organism in 4 horses.

Coagulopathy in adult horses with infection or inflammation has been well documented. Horses presenting for evaluation for colic were found to have at least one abnormal coagulation parameter, with APTT prolonged in 63% of cases and PT prolonged in 25% of cases. (Johnstone & Crane, Equine Vet J, 18(4), 271-274, 1986) In a case series of six horses suffering from DIC, prolonged APTT and PT were found in horses with regional or generalized infection, neoplasia, or ulcerative colitis. (Morris & Beech, J Am Vet Med Assoc, 183(10), 1067-1072, 1983) Pleuropneumonia and gastrointestinal disease have been demonstrated to be associated with consumption of clotting factors leading to prolongation of clotting times, including PT and APTT. (Prasse, et al., J Am Vet Med Assoc, 203(5), 685-693, 1993; Dallap, et al., Vet Emerg Crit Care, 13(4), 215-225, 2003; Epstein, et al., J Vet Intern Med, 25(2), 307-314, 2011; Tomlinson, et al., 29(5), 1410-1417, 2015) Coagulopathies have been identified in association with pneumonia in horses caused by Klebsiella sp., Actinobacillus equuli, and S. zooepidemicus. (Yoshikawa, et al., J Vet Med Sci, 65(7), 787-792, 2003; Pusterla, et al., J Vet Intern Med, 21(2), 344-347, 2007; Pusterla, et al., J Vet Diagn Invest, 20(1), 118-121, 2008; Estell, et al., J Vet Intern Med, 30(1), 314-321, 2016).

TABLE 8
Clinical Signs of Pneumonia

Treatment Group

| Clinical Sign | 0X | 1X | 2X | 3X |
|---|---|---|---|---|
| Cough | | 1 | 2 | 1 |
| Depression | 2 | 2 | 3 | 3 |
| Tachypnea and/or Dyspnea | | | 1 | 2 |
| Epistaxis | | | | 2* |
| Nasal Discharge | | 1 | 2 | 1 |
| Inappetence | 1 | 2 | 3 | 3 |
| Febrile | 1 | 1 | | |

0X, placebo;
1X, 30 mg/kg dipyrone;
2X, 60 mg/kg dipyrone;
3X, 90 mg/kg dipyrone;
*Discharge was typically seromucoid and not bloody.

During the study eleven horses dispersed across all treatment groups developed clinical signs of mild to severe pneumonia. The majority of the remaining clinically normal horses were diagnosed with minimal to advanced pneumonia at necropsy with corresponding clinical pathology findings representing an inflammatory leukogram.

### Example 2: Evaluation of Coagulation Effects and Pharmacokinetics of Metamizole Administered Intravenously Twice or Three Times Daily in Horses

The purpose of this study was to evaluate the effects of metamizole on coagulation parameters and pharmacokinetic parameters when administered intravenously to horses for 9 days at doses of 0 mg/kg (0×), 30 mg/kg (1×), 60 mg/kg (2×), and 90 mg/kg (3×) of body weight. Doses of 0×, 1×, 2×, and 3× were administered twice daily (BID), every 12 hours±120 min. Doses of 1× and 2× were administered three times daily (TID), every 8 hours±120 min. Metamizole plasma concentrations were assessed in the 1×BID, 3×BID, 1×TID, and 2×TID treatments groups to evaluate for evidence of accumulation.

### Trial Design

This was a prospective, randomized, masked, placebo-controlled, parallel-group laboratory study. The protocol was approved by the research laboratory's Institutional Animal Care and Use Committee (IACUC). The study was conducted in compliance with the principles of the relevant CVM Good Clinical Practices Guidelines. Horse care and housing was in compliance with Guide for the Care and Use of Agricultural Animals in Research and Teaching. The study was conducted at a research laboratory with extensive experience in regulatory studies of large animals in the United States.

### Horses and Acclimation

Horses were acquired from a single ranch and assembled in a herd at the research facility for two weeks of acclimation. Forty adult horses were evaluated for participation in the study. Inclusion criteria included age between 3 and 20 years, weight between 350 and 650 kg, bilateral patent jugular veins, being amenable to the study procedures, and no known history of medication use within 14 days of study protocol initiation. Exclusion criteria included pregnancy, lactation, ill thrift, and a known history of NSAID sensitivity.

Horses were housed in individual pens within a covered barn. Diet consisted of hay fed two to three times daily, a concentrate fed once daily, and water ad libitum. Horses were given unique identification collars and microchipped. Due to the presence of indwelling intravenous catheters pasture turnout was not permitted.

### Masking and Randomization

The Clinical Investigator, veterinarians, clinical pathology laboratory, and site personnel involved in data collection and observations were masked to treatment allocation throughout the study. Dose administrators and the statistician were not masked and did not participate in data collection. Masking was maintained until the completion of data collection and locking of the database.

Thirty-two horses were randomly allocated to one of six treatment groups (Table 9).

TABLE 9
Treatment Groups

| | | | | | Total | |
|---|---|---|---|---|---|---|
| | | | | | Dose per | |
| | | Number | IV Dosage | | Day | Dose |
| Group | Treatment | (Male/Female) | (mg/kg) | Dose Frequency | (mg/kg) | Level |
| 1 | 0.9% NaCl | 2/2 | 0 | BID | 0 | 0X |
| 2 | Dipyrone | 3/3 | 30 | BID | 60 | 1X |
| 3 | Dipyrone | 3/3 | 60 | BID | 120 | 2X |
| 4 | Dipyrone | 3/3 | 90 | BID | 180 | 3X |
| 5 | Dipyrone | 2/2 | 30 | TID | 90 | 1X |
| 6 | Dipyrone | 3/3 | 60 | TID | 180 | 2X |

IV = intravenous

### Treatment Phase (Investigational Drug and Placebo)

The investigational drug was a sterile injectable formulation of dipyrone (Zimeta®, Corden Pharma, Caponago, Italy) (500 mg/mL) in 100 mL glass bottles. The placebo was sterile injectable saline (0.9% Saline, Nova-Tech Inc, Grand Island, NE) (0.9% sodium chloride) in 500 mL plastic bottles labeled for veterinary use. Dipyrone treatment groups were 30 mg/kg/dose (1×) administered twice daily (BID), 1× administered three times daily (TID), 60 mg/kg/dose (2×) BID, 2×TID, and 90 mg/kg/dose (3×) BID. Placebo treatment group, (0×) BID, were saline administered at a volume to match the 1× dipyrone group. The dose was calculated based on body weight and administered for nine consecutive days (Days 1-9). Due to the large volume of the injection at the higher test intervals, treatment was administered via indwelling intravenous catheters. Intravenous catheters were placed using standard techniques. Horses were sedated with xylazine (XylaMed™, VetOne, MWI, Boise, Id.) (0.6 mg/kg, IV) and the skin was desensitized with subcutaneous injection of lidocaine (Lidocaine 2%, VetOne, MWI, Boise, Id.). Intravenous catheters were replaced at the discretion of the study director and were maintained with heparinized saline flush prior to, and following, dose administration.

Horses were acclimated to the facility for 14 days prior to start of the treatment phase. During acclimation, all horses underwent a physical examination, nasal lavage culture to rule out Streptococcus equi subsp. equi (S. equi), and blood collection for clinical pathology (hematology, serum chemistry, and serial coagulation panels). On Day −1, thirty-two horses were randomized by the study statistician and moved to their assigned pens. On Day −1 (baseline), Day 4, and Day 10 horses had blood collected for a coagulation panel. A physical exam was performed prior to study start and on Day 10. Horses were dosed according to randomization beginning on Day 1 and were observed twice daily for clinical signs.

### Statistics

All statistical analyses were performed using SAS, version 9.4 (SAS Institute, Cary, N.C.). The individual horse was the experimental unit in the statistical analysis. For statistical modeling, all main effect, interaction terms, and pair-wise comparisons of the active groups (dipyrone) with the placebo group were assessed at the two-sided 0.10 significance level (unadjusted), except for the evaluation of the three-way interaction which was assessed at the two-sided 0.05 significance level (unadjusted).

For continuous endpoints with repeated post-treatment measurements (coagulation), repeated measurement analysis of covariance (SAS MIXED procedure repeated measures of analysis of covariance (RMANCOVA)) were used to evaluate a model including treatment, time, sex, treatment by sex interaction, treatment by time interaction, time by sex interaction, and treatment by time by sex three-way interaction as fixed effects. In addition, baseline values were included as a covariate and remained in the model regardless of its significance. Baseline values were defined as the values prior to and nearest to the first dosing. The covariance structures for repeated measures analysis of covariance (RMANCOVA) were selected from compound symmetry (CS) and compound symmetry heterogeneous (CSH) variances, since only two post baseline measurements were collected in this study. The Kenward-Roger approximation was used to calculate denominator degrees of freedom for statistical tests. The minimum Akaike Information Criterion was used to select the covariance structure. The selection of covariance structure was based on PT. The resulting covariance structure was then used in the modeling of all coagulation endpoints.

If the 3-way interaction (treatment by time by sex) was significant, the statistical analysis was considered inconclusive given the small sample size, and no further statistical analyses were conducted. If the 3-way interaction was not significant, treatment by time and treatment by sex interactions were evaluated. If the treatment by time interaction was significant (regardless of significance of treatment by sex interaction), within time pair-wise comparisons of each treatment group versus placebo using linear contrasts were performed. If the treatment by sex interaction was significant (regardless of significance of treatment by time interaction), within sex pair-wise comparisons of each treatment group versus placebo using linear contrasts were performed.

If neither of the two-way interactions involving treatment were significant, the main effect of treatment was evaluated. If the treatment effect was significant, pair-wise comparisons of each treatment group versus placebo using linear contrasts were performed.

### Results

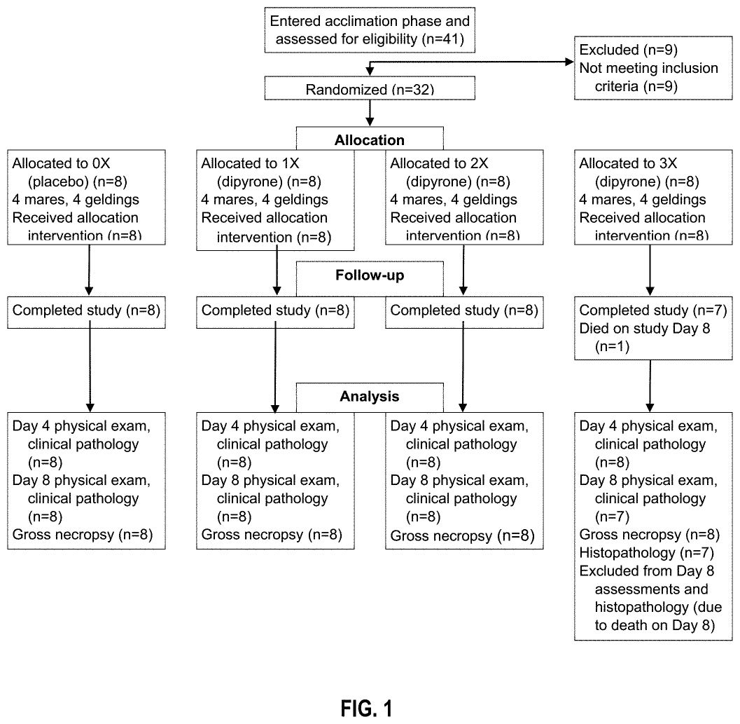

Thirty-two horses met the enrollment criteria and were randomized to treatment (FIG. 1, Table 9). Horses ranged in age from 3-20 years and weighed between 388 and 627 kg. Quarter horses and Draft horse were included. All horses completed the study. Masking was intact throughout the study.

Horses were acclimated to the facility for 14 days prior to start of the treatment phase. During acclimation, all horses underwent a physical examination, nasal lavage culture to rule out Streptococcus equi subsp. equi (S. equi), and blood collection for clinical pathology (hematology, serum chemistry, and serial coagulation panels). On Day −1, thirty-two horses were randomized by the study statistician and moved to their assigned pens. On Day −1 (baseline), Day 4, and Day 10 horses had blood collected for a coagulation panel. A physical exam was performed prior to study start and on Day 10. Horses were dosed according to randomization beginning on Day 1 and were observed twice daily for clinical signs.

### Observations

All 32 horses were acclimated prior to study start and started the treatment phase healthy. Few horses presented with abnormal clinical signs in the treatment period and none were considered treatment related (Table 10). In total, 14 horses had observations reported for catheter-related changes to the jugular vein, including phlebitis (Table 10). Samples were collected from the IV catheter or insertion site exudate at the final catheter removal from six horses and submitted for culture and sensitivity (IDEXX Preclinical Research Services, West Sacramento, Calif.). Five horses showed bacterial growth. The specific organisms cultured included three Staphylococcus spp., Actinobacillus sp., Acinetobacter sp., and Klebsiella sp. The three Staphylococcus spp. isolated were methicillin resistant and were considered commensal organisms of the skin.

TABLE 10
Clinical Signs in Treated Horses

| | Treatment Group | | | | | |
|---|---|---|---|---|---|---|
| | 0X BID | 1X BID | 1X TID | 2X BID | 2X TID | 3X BID |
| Clinical Signs | (n = 4) | (n = 6) | (n = 4) | (n = 6) | (n = 6) | (n = 6) |
| Phlebitis and other catheter- | 3 | 2 | 1 | 3 | 2 | 3 |
| related changes to jugular | | | | | | |
| vein | | | | | | |
| Minor respiratory signs (nasal | 1 | 1 | 0 | 0 | 0 | 1 |
| discharge and or cough) | | | | | | |
| Minor gastrointestinal signs | 0 | 2 | 1 | 2 | 2 | 1 |
| or inappetence | | | | | | |
| Suture site abscesses | 0 | 1 | 0 | 0 | 1 | 0 |
| Ocular discharge | 0 | 0 | 0 | 1 | 0 | 0 |
| Depression | 0 | 0 | 0 | 0 | 1 | 0 |
| Dermatitis | 0 | 0 | 0 | 0 | 0 | 1 |

Abnormal clinical signs in treated horses.
0X BID, placebo;
1X, 30 mg/kg dipyrone;
2X, 60 mg/kg dipyrone;
3X, 90 mg/kg dipyrone,
BID, twice daily,
TID, three times daily.

### Concomitant Medications

Intravenous catheters were placed by site veterinarians following standard veterinary technique, including sedation with xylazine and local tissue block with lidocaine. No additional medication was administered in the treatment period. Following completion of all study activities horses were administered an antibiotic; ceftiofur crystalline free acid (Excede®, Zoetis, Pharmacia & Upjohn Company, NY, NY) (6.6 mg/kg, IM) to treat the catheter related phlebitis.

Thrombophlebitis is a known sequela to the use of IV catheters. Recommendations to minimize the incidence of thrombophlebitis; including reducing prolonged IV catheter placement, minimizing concurrent diseases, and avoiding irritation of the vein, are challenging to fulfill in a research scenario. Research horses in this study displayed active irritation towards the IV catheters including vigorous attempts to rub the catheters. The IV catheters remained wrapped for the duration of the study, which prevented horses from easily removing the IV catheter, but also increased local irritation at the catheter site and made early detection of phlebitis difficult. NSAID treatment is commonly recommended for horses with thrombophlebitis. The use of dipyrone may have minimized the severity of thrombophlebitis in some of the treated horses. Studies evaluating the development of thrombophlebitis in horses with IV catheters reported high rates (18%-29% horses) and increased frequency of thrombophlebitis in horses with catheters in place for a longer duration. Bacteria isolated from IV catheter sites in this study are consistent with previous reports. Due to the constraints of the study, the horses were not treated with recommended antimicrobials during the study but were treated for ethical reasons following the end of the study procedures.

Prothrombin time measures the activities of the extrinsic and common coagulation pathway factors.

TABLE 11
Prothrombin Time - All Groups

| | Treatment | | | |
|---|---|---|---|---|
| Parameter | Group | LSMEAN | SEM | P-value |
| Prothrombin Time | 0X BID | 12.977 | 0.2394 | — |
| | 1X BID | 13.407 | 0.2014 | 0.187 |
| | 1X TID | 13.553 | 0.2419 | 0.106 |
| | 2X BID | 13.469 | 0.1958 | 0.128 |
| | 2X TID | 13.733 | 0.1965 | 0.025 |
| | 3X BID | 14.238 | 0.1974 | <0.001 |

0X BID, placebo;
1X, 30 mg/kg dipyrone;
2X, 60 mg/kg dipyrone;
3X, 90 mg/kg dipyrone,
BID, twice daily,
TID, three times daily;
LS mean, Least Square mean;
SEM, standard error of the mean;
n/a, non-applicable.
p-values are statistically significant at p < 0.1.

TABLE 12
Prothrombin time - High Dose Groups

| | | | | Difference in |
|---|---|---|---|---|
| | | | | Individual |
| | Prothrombin Time (sec) | | | Horse Day |
| Treatment | Baseline | | | 10 PT and |
| Group | Range | Day 4 Range | Day 10 Range | Baseline PT† |
| 0X BID | 12.0-14.8 | 12.4-13.1 | 12.7-13.4 | 0 |
| 3X BID | 11.9-14.7 | 13.2-14.5 | 13.4-14.9 | 0.1-0.7 sec |
| 2X TID | 11.9-16.1 | 12.9-15.6 | 13.2-16.6 | 0.1-0.5 sec |

†defined as the difference between the Day 10 PT value and the highest baseline PT value.
N/A (non-applicable) is for horses where the Day 10 PT value was within the baseline PT range.
PT, prothrombin time

For the area under the curve (AUC), ANOVA (the GLM procedure in SAS) was used to evaluate differences between the treatment groups. The model included the following fixed effects: treatment (TRT), sex (SEX) and the two-way interaction TRT*SEX. For AUC, the CA group was not included in the analysis. If the TRT*SEX interaction was significant, treatment means were compared to each other for each individual sex. An all pair-wise comparison approach was used and differences evaluated using an unadjusted alpha=0.10. If the interaction was not significant and the TRT main effect was significant, treatment means were compared to each other pooled across sex. An all pair-wise comparison approach was used and differences evaluated using an unadjusted alpha=0.10. If the interaction term was not significant and the TRT main effect was not significant, no further evaluations were conducted.

For the coagulation outcomes, a repeated measurement analysis of covariance (SAS MIXED procedure RMANCOVA) was used to evaluate a model including treatment, time, sex, treatment by sex interaction, treatment by time interaction, time by sex interaction, and treatment by time by sex three-way interaction as fixed effects. In addition, baseline values were included as a covariate and remained in the model regardless of its significance. Baseline values were defined as the values prior to and nearest to the first dosing.

### Pharmacokinetic Analysis

Samples for pharmacokinetic analysis were collected as indicated in Table 13 below.

TABLE 13
Pharmacokinetic Sampling Schedule

| Dose | Study Day | | | | | | | | |
|---|---|---|---|---|---|---|---|---|---|
| Group | 1 | 2 | 3 | 4 | 5 | 6 | 7 | 8 | 9 |
| 2 (1X | Baseline, † | Trough 1 | NS | Trough 1 | NS | Trough 1 | NS | Trough 1 | Trough |
| BID) | | | | | | | | | 1, |
| | | | | | | | | | Trough |
| | | | | | | | | | 2, † |
| 4 (3X | Base- | Trough 1 | NS | Trough 1 | NS | Trough 1 | NS | Trough 1 | Trough |
| BID) | line, 15 min, | | | | | | | | 1, |
| | Trough 2 | | | | | | | | Trough 2 |
| 5 (1X | Baseline, ‡ | Trough 1 | NS | Trough 1 | NS | Trough 1 | NS | Trough 1 | Trough |
| TID) | | | | | | | | | 1, |
| | | | | | | | | | Trough |
| | | | | | | | | | 2, |
| | | | | | | | | | Trough |
| | | | | | | | | | 3, ‡ |
| 6 (2X | Base- | Trough 1 | NS | Trough 1 | NS | Trough 1 | NS | Trough 1 | Trough |
| TID) | line, 15 min, | | | | | | | | 1, |
| | Trough 3 | | | | | | | | Trough |
| | | | | | | | | | 2, |
| | | | | | | | | | Trough 3 |

† 15 min, 1, 4, 8, and 12 hours (samples scheduled for Day 9 were collected following the 1^{st} dose of the day)
‡ 15 min, 1, 4, and 8 hours (samples scheduled for Day 9 were collected following the 1^{st} dose of the day)
NS—no sample collected

Plasma samples were analyzed for 4-methylamino antipyrine (4-MAA) using an analytical method developed and validated by Pyxant Labs Inc. over the range of 150 to 25,000 ng/mL. Samples were prepared by a protein precipitation extraction procedure. Extracts were analyzed by liquid chromatography/tandem mass spectrometry (LC/MS/MS) in positive ionization mode under optimized conditions for detection of 4-MAA and 4-MAA-d3 positive ions formed by electrospray ionization.

Pharmacokinetic parameters for the active metabolite of dipyrone (4-MAA) were determined using noncompartmental analysis over a range of doses and dosing intervals. Commercially available software (Phoenix WinNonlin® ver 7.0, Pharsight, Cary, N.C.) was used for the analysis. An extravascular dosing model was chosen for analysis of the active metabolite. As a measure of total systemic drug exposure, the area under the curve for the treatment interval (AUG₀₋ₗₐₛₜ) was determined for each dosage regimen using the linear trapezoidal rule using the equation: Σi(ti−ti−1)(Ci+Ci−1)/2, i=1 to t, where Ci is the sample concentration at time ti. The accumulation index over all dose ranges was calculated using the equation: 1/(1−e^{-λzτ}). Dose proportionality was determined by using the maximum concentration detected following the initial drug administration (0.25 h) and correcting this value for dose (Cₘₐₓ/D). Additional parameters, including steady state clearance (Clₛₛ/F) and AUG_{0-τ} were calculated for the 1×BID and 1×TID dose groups.

A one-way ANOVA was used to determine significant differences between parameters among the different dosage regimens. Significance was determined at P<0.05. The linearity across doses was determined using linear regression analysis (Sigmaplot® ver 13.0, Systat Software, San Jose, Calif.).

To ensure that changes in APTT and PT post-treatment were applicable to the individual horse, this study used three pre-treatment coagulation samples as the reference point. Three pre-study coagulation samples were collected from each horse to ensure that changes in APTT and PT post-treatment could be compared to each animal's own individual baseline value.

No main treatment effect was found for activated partial thromboplastin time (APTT) or fibrinogen across all treatment groups (data not shown).

### Pharmacokinetics

Three time points were excluded from the analysis due to aberrant results. One horse in the 1×BID group (horse 15) had an abnormally high trough concentration at sample day 4, which was due to an overdose of drug received on sample day 3. Two horses in the 3×BID group had abnormally low concentrations detected on sample day 4 (horse 17) or sample day 1 (horse 6). These samples were most likely dilution errors or sampling errors and therefore were excluded from analysis. This ensured that the 3×BID group calculations were not lower due to laboratory or handling errors.

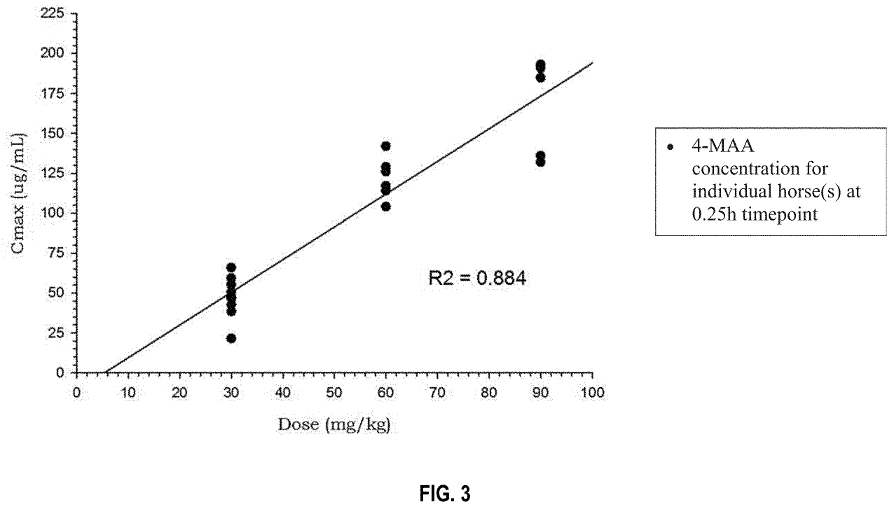

Total systemic exposure of 4-MAA, as determined by the AUC₀₋ₗₐₛₜ increased as the dose increased or the dosing interval decreased. The accumulation index increased as the dose increased or the dosing interval decreased (Table 14). Statistically significant differences were noted among groups. The 4-MAA concentrations were proportionate and linear over a range of 1×-3×. Using data from the first dose administered, the concentration measured at the first time point (0.25 h) was linear over the range of 30-90 mg/kg (R²=0.884) (FIG. 3).

TABLE 14
Accumulation Index

| Treatment Group | Horse ID | Accumulation Index |
|---|---|---|
| 1X BID | 15 | 1.39 |
| 1X BID | 18 | 1.17 |
| 1X BID | 25 | 1.08 |
| 1X BID | 26 | 1.05 |
| 1X BID | 39 | 1.20 |
| 1X BID | 7 | 1.26 |
| 1X TID | 19 | 2.30 |
| 1X TID | 23 | 2.62 |
| 1X TID | 32 | 2.57 |
| 1X TID | 5 | 2.88 |
| 2X TID | 1 | 9.68 |
| 2X TID | 12 | 13.56 |
| 2X TID | 27 | 7.46 |
| 2X TID | 3 | 15.86 |
| 2X TID | 36 | 7.70 |
| 2X TID | 37 | 28.51 |
| 3X BID | 17 | 3.23 |
| 3X BID | 21 | 7.50 |
| 3X BID | 29 | 15.81 |
| 3X BID | 30 | 4.36 |
| 3X BID | 4 | 21.19 |
| 3X BID | 6 | 3.52 |

TABLE 15
Dose Proportionality

| Treatment Group | Horse ID | Cmax | Cmax/Dose |
|---|---|---|---|
| 1X BID | 15 | 46.5 | 1.6 |
| 1X BID | 18 | 38.5 | 1.3 |
| 1X BID | 25 | 47.1 | 1.6 |
| 1X BID | 26 | 47.3 | 1.6 |
| 1X BID | 39 | 21.6 | 0.7 |
| 1X BID | 7 | 42.7 | 1.4 |
| 1X TID | 19 | 55.3 | 1.8 |
| 1X TID | 23 | 50.6 | 1.7 |
| 1X TID | 32 | 59.3 | 2.0 |
| 1X TID | 5 | 65.9 | 2.2 |
| 2X TID | 1 | 142 | 2.4 |
| 2X TID | 12 | 104 | 1.7 |
| 2X TID | 27 | 129 | 2.2 |
| 2X TID | 3 | 114 | 1.9 |
| 2X TID | 36 | 117 | 2.0 |
| 2X TID | 37 | 126 | 2.1 |
| 3X BID | 17 | 132 | 1.5 |
| 3X BID | 21 | 193 | 2.1 |
| 3X BID | 29 | 136 | 1.5 |
| 3X BID | 30 | 185 | 2.1 |
| 3X BID | 4 | 191 | 2.1 |
| 3X BID | 6 | 15.6 | 0.2* |

*excluded from linear regression

Comparison of the 1×BID dose to the 1×TID dose revealed no significant difference between clearance (Clss/F) and AUC_{0-τ}. Significant differences were found in other parameters, including an increased minimum concentration (Cₘᵢₙ), and average concentration (C_{avg}) over the treatment period for the TID dosage regimen (P<0.001). Half-life also increased with TID dosing, due to an increased volume of distribution (P<0.001) (Table 16).

TABLE 16
Pharmacokinetic Parameter Calculations for 1X Dose Groups

| Parameter | 1X BID* | 1X TID* | P-value |
|---|---|---|---|
| Clss/F (mL/kg/min) | 4.72 ± 0.60 | 4.05 ± 0.52 | 0.11 |
| AUC tau (hr * ug/mL) | 107.16 ± 12.18 | 124.91 ± 15.22 | 0.074 |
| Cmin (μg/mL) | 2.47 ± 0.59 | 5.89 ± 1.11 | <0.001 |
| Cavg (μL/mL) | 8.93 ± 1.02 | 15.61 ± 1.90 | <0.001 |
| Half-life (h) | 4.48 ± 1.37 | 11.37 ± 1.33 | <0.001 |
| Vz/F (L/kg) | 1.81 ± 0.54 | 3.97 ± 0.58 | <0.001 |

*Mean and standard deviation

### Example 3: Two Studies Evaluating of Efficacy of Metamizole for Treatment of Pyrexia in Horses

### Materials and Methods

Study 1: The unmasked laboratory study was conducted at a clinical research organization in the United States. Institutional Animal Care and Use Committee (IACUC) approval was obtained prior to the conduct of the study. Horses one year of age or older were comingled by the Investigator in order to facilitate transmission of respiratory infections. Horses were monitored by the study staff for elevation of rectal temperature. All horses received treatment once the rectal temperature was ≥102.0° F. A single dose of dipyrone was administered intravenously (30 mg/kg IV). Rectal temperature was monitored 1, 2, 3, and 6 hours post-treatment administration.

Study 2: The masked, randomized, placebo controlled laboratory study was conducted at a clinical research organization in the United States. IACUC approval was obtained prior to the conduct of the study. Horses one year of age or older were comingled by the Investigator in order to facilitate transmission of respiratory infections. Horses were monitored by the study staff for elevation of rectal temperature. Once the horse reached a rectal temperature of ≥102.0° F. it was randomized to treatment in a 1:1 ratio (dipyrone:saline) in order of presentation. Unblinded dose administrators administered the study drug (Phase 1). A single dose of study drug was administered. Treatment was either dipyrone (30 mg/kg IV) or placebo (saline, matched volume, IV). Blinded personnel recorded the rectal temperature at 4 and 6 hours post-treatment administration. Twenty-four hours following Dose 1 horses were monitored for rectal temperature. Horses with rectal temperature≥102.0° F. were crossed over to the other treatment group. Unblinded dose administrators administered the study drug (Phase 2), and blinded personnel recorded the rectal temperature 4 and 6 hours post-treatment administration. Horses with a normal temperature 24 hours after Dose 1 did not receive a second treatment. Blinding was maintained until the completion of the statistical analysis.

In both studies horses were assessed daily to determine overall safety. Medications that could have impacted the temperature or the underlying cause of the disease were not permitted in either study; however, horses in both studies were eligible for rescue at the discretion of the Investigator. Following completion of the study, the Investigator treated the horses with standard of care for the type, and severity, of clinical signs. Statistical analysis was performed with the Wilcoxon rank sum test (SAS®, SAS Institute, Cary N.C., version 9.4).

### Results

Study 1: A total of eight horses met the enrollment criteria and were enrolled in the study. Enrolled breeds were Tennessee Walking Horses (n=4), Quarter Horses (n=2), grade (n=1), and Paint Horse (n=1). The population was comprised of five geldings and three mares, with an age range of three to 20 years, as estimated by dentition. Dipyrone reduced fever (>1° F.) in seven horses (87.5%), with a return to normothermia (≥2° F.) in six horses (75%) at Hour 6. Dipyrone significantly reduced fever at all measured timepoints post-dose (Table 17). Few abnormal clinical signs were identified and were consistent with respiratory infections.

TABLE 17
Temperature Change in Study 1

| | Mean (± SD) temperature change | |
|---|---|---|
| Parameter | from baseline (° F.) | P-value |
| Hour 1 | −1.1 (± 0.5) | 0.0078 |
| Hour 2 | −1.4 (± 1.0) | 0.0156 |
| Hour 3 | −1.9 (± 1.0) | 0.0156 |
| Hour 6 | −1.9 (± 1.1) | 0.0078 |

Study 2: A total of 31 horses met the enrollment criteria and were treated. Enrolled breeds included Paint Horses, Quarter Horses, and Tennessee Walking Horses. Twenty-one mares and ten geldings were enrolled. Horses ranged in age from three to twenty years of age, as estimated by dentition. In Phase 1, 15 horses were treated with dipyrone and 16 horses were treated with placebo (saline). In Phase 2 a total of 27 horses qualified for treatment, and 4 horses were afebrile and did not receive treatment. In Phase 2, 14 horses were treated with dipyrone and 13 horses were treated with placebo (saline).

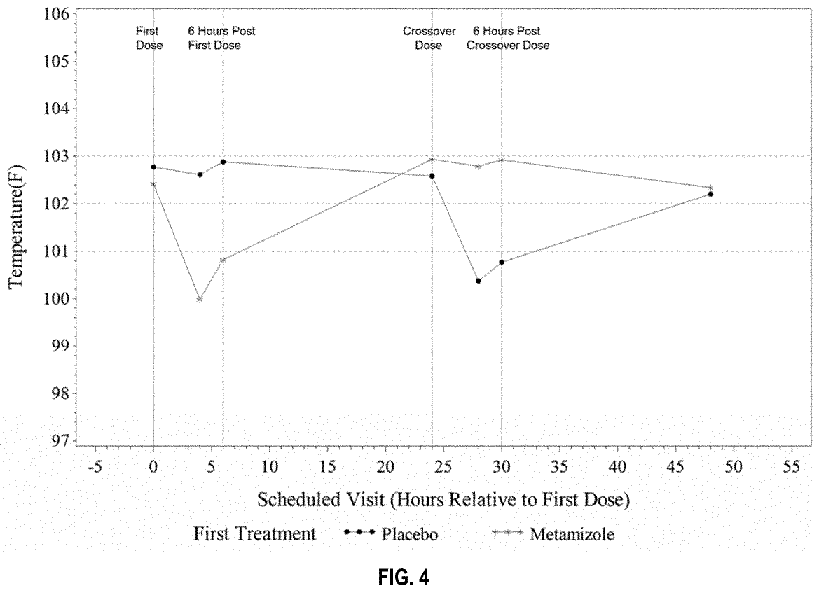

The mean (standard deviation) temperature in Phase 1 at hour 4 was 102.6° F. (1.4) in the placebo group and 100.0° F. (0.9) in the dipyrone group. By hour 6, the mean temperature was 102.9° F. (1.4) in the placebo group and 100.8° F. (1.3) in the dipyrone group. The mean temperature in Phase 2 at hour 4 was 102.8° F. (0.8) in the placebo group and 100.4° F. (0.8) in the dipyrone group. By hour 6 the mean temperature was 102.9° F. (1.1) in the placebo group and 100.8° F. (1.0) in the dipyrone group. The maximum decrease in rectal temperature was found at hour 4 in comparison to hour 6 (FIG. 4). In both phases, dipyrone significantly lowered rectal temperatures in treated horses compared to controls at Hour 4 (p<0.0001).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating an equine with pyrexia, said method comprising administering a therapeutically effective dose of metamizole to said equine, wherein the administration is repeated at least once after an interval of 12 hours.

2. The method of claim 1, wherein said dose is in the range of 20 mg metamizole per kg of equine subject weight (mg/kg) to 50 mg/kg.

3. The method of claim 2, wherein said dose is 30 mg metamizole per kg of equine subject weight (mg/kg).

4. The method of claim 1, wherein the metamizole is administered twice a day for at least 2 days.

5. The method of claim 1, wherein the metamizole is administered no more than 18 times in a period of 9 days.

6. The method of claim 1, wherein the dose is administered intramuscularly, intravenously (IV), or subcutaneously.

7. The method of claim 6, wherein the dose is administered intravenously.

8. The method of claim 1, wherein the metamizole is comprised within a pharmaceutical formulation.

9. The method of claim 8, wherein the concentration of metamizole in the pharmaceutical formulation is from 200 mg/ml to 750 mg/ml.

10. The method of claim 9, wherein the concentration of metamizole in the pharmaceutical formulation is from 400 mg/ml to 600 mg/ml.

11. The method of claim 10, wherein the concentration of metamizole in the pharmaceutical formulation is 500 mg/ml.

12. The method of claim 1, wherein the metamizole is metamizole sodium monohydrate.

13. The method of claim 1, wherein the pyrexia is due to a condition selected from viral infections, bacterial infections, parasitic infections, respiratory infections, tick-borne infections, mosquito-borne infections, equine encephalomyelitis, equine influenza, equine herpes virus, West Nile virus, strangles, Potomac horse fever, colic, post-vaccination reactions, physical trauma, and stress.

14. A method of treating pyrexia in an equine via a dosage regimen comprising administering metamizole to the equine in the range of 20 mg metamizole per kg of equine subject weight (mg/kg) to 50 mg/kg, at a dosing interval of no more than twice daily.

15. The method of claim 14, wherein the metamizole is administered in an amount of 30 mg/kg no more than twice daily.

16. The method of claim 14, wherein the dose is administered intramuscularly, intravenously (IV), or subcutaneously.

17. The method of claim 16, wherein the dose is administered intravenously.

18. The method of claim 14, wherein the metamizole is administered to the equine for at least 2 days.

19. (canceled)

20. The method of claim 14, wherein the metamizole is comprised within a pharmaceutical formulation.

21. (canceled)

22. The method of claim 20, wherein the concentration of metamizole in the pharmaceutical formulation is from 400 mg/ml to 600 mg/ml.

23. The method of claim 22, wherein the concentration of metamizole in the pharmaceutical formulation is 500 mg/ml.

24. The method of claim 14, wherein the metamizole is metamizole sodium monohydrate.

25. The method of claim 14, wherein the pyrexia is due to a condition selected from viral infections, bacterial infections, parasitic infections, respiratory infections, tick-borne infections, mosquito-borne infections, equine encephalomyelitis, equine influenza, equine herpes virus, West Nile virus, strangles, Potomac horse fever, colic, post-vaccination reactions, physical trauma, and stress.
